(19) United States
(12) Patent Application Publication
(10) Pub. No.: US 2024/0008752 A1
(43) Pub. Date: Jan. 11, 2024

(54) BIOMETRIC DATA MEASUREMENT SYSTEM

(71) Applicant: Murata Manufacturing Co., Ltd., Nagaokakyo-shi (JP)

(72) Inventor: Toru SHIMUTA, Nagaokakyo-shi (JP)

(21) Appl. No.: 18/470,912

(22) Filed: Sep. 20, 2023

Related U.S. Application Data
(63) Continuation of application No. PCT/JP22/16610, filed on Mar. 31, 2022.

(30) Foreign Application Priority Data
Apr. 30, 2021 (JP) 2021-077303

(51) Int. Cl. A61B 5/021; A61B 5/00

(52) U.S. Cl. CPC A61B 5/021; A61B 5/6826; A61B 5/7425; A61B 5/0077; A61B 5/6843; A61B 5/0022; A61B 2560/0406; A61B 2560/0431

(57) ABSTRACT

A biometric data measurement system is provided that includes an annular biometric sensor and a portable control unit that communicate with each other. The portable control unit determines whether or not the portable control unit is gripped by a hand on which the annular biometric sensor is mounted, determines whether or not a measurement posture of a user is sufficient from a position of a face of the user in an image recognized from the image, and an inclination of the portable control unit from a vertical direction, controls the biometric data measurement system based on a gripping determination result and a posture determination result, and acquires biometric data including blood pressure.

### CROSS REFERENCE TO RELATED APPLICATIONS

This application is a continuation of International Application No. PCT/JP2022/016610, filed Mar. 31, 2022, which claims priority to Japanese Patent Application No. 2021-077303, filed Apr. 30, 2021, the entire contents of each of which are hereby incorporated by reference in their entirety.

### TECHNICAL FIELD

The present invention relates to a biometric data measurement system.

### BACKGROUND

When a measurement site of the blood pressure is located higher than the heart, the measurement value of the blood pressure is lower by the pressure difference of hydrostatic pressure in a blood vessel due to gravity. Conversely, when the measurement site of the blood pressure is located lower than the heart, the measurement value of the blood pressure is higher by the pressure difference of hydrostatic pressure in the blood vessel. More specifically, when the measurement site of the blood pressure moves up and down from the height of the heart by 1 cm, the blood pressure (e.g., the measurement value) changes by about 0.7 mmHg.

As an example, Japanese Unexamined Patent Application Publication No. 2009-247733 (hereinafter “Patent Document 1”) discloses an electronic sphygmomanometer including a camera that has a predetermined image capturing range, performs an image capturing operation when blood pressure is measured, and outputs image data, a face and cuff detection unit that detects whether or not a captured image indicated by the image data includes an image of a face and an image of a cuff, based on the image data, position information calculation unit that calculates position information of the image of the face and the image of the cuff in the captured image, a fraud determination unit that executes a process of determining whether or not a use state of the electronic sphygmomanometer is appropriate, based on a relative positional relationship indicated by the calculated position information of the image of the face and the image of the cuff, and an output unit that outputs a result of the determination by the fraud determination unit. According to this electronic sphygmomanometer, when the blood pressure is measured, it is determined whether or not the use state of the electronic sphygmomanometer is appropriate, based on the relative positional relationship between the image of the face and the image of the cuff in the captured image. Thus, it is possible to detect whether the use state of the electronic sphygmomanometer is appropriate, for example in terms of measurement accuracy.

Moreover, Japanese Unexamined Patent Application Publication No. 2020-500052 (hereinafter “Patent Document 2”) has proposed a device including a blood pressure sensor that acquires a blood pressure measurement value from a user holding the device with a hand, and a control unit that determines an angle of the device with respect to a gravity direction, specifies one or a plurality of positions of the user holding the device with the hand, within a predetermined displayed position range on a display screen of the user, determines a height of the blood pressure sensor with respect to a height of the heart of the user based on the angle of the device with respect to the gravity direction and the one or plurality of positions of the user in an image within the predetermined position range, and performs control based on the height of the blood pressure sensor with respect to the height of the heart of the user. According to this device, it is possible to control the device and measure blood pressure based on the height of the blood pressure sensor with respect to the height of the heart of the user.

However, the electronic sphygmomanometer disclosed in Patent Document 1 is invasive because the cuff tightens the upper arm when measuring the blood pressure. In addition, since the cuff is used, the size of the device (e.g., the electronic sphygmomanometer) is large, and the device is unsuitable for portability. Therefore, for example, it is not possible to use the device to measure blood pressure on the go.

On the other hand, in the device disclosed in Patent Document 2, since the device is held with the hand and the finger of the hand is brought into contact with the blood pressure sensor to measure the blood pressure, it is difficult to keep the contact pressure constant. When the contact pressure changes, the blood pressure of the measurement site being in contact fluctuates, i.e., there are fluctuations between measurements but contact pressure fluctuations are particularly likely to occur during measurement. Thus, there is a concern that it is difficult to stably measure the blood pressure.

### SUMMARY OF THE INVENTION

Accordingly, it is an object of the present invention to provide a biometric data measurement system that is excellent in portability and is configured to measure biometric data including blood pressure, whose measurement value is influenced by a difference between the height of the measurement site and the height of the heart (that is, influenced by hydrostatic pressure), in a non-invasive manner with higher accuracy.

According to an exemplary aspect, a biometric data measurement system is provided that includes an annular biometric sensor and a portable control unit that are configured to communicate with each other. The annular biometric sensor includes a main body formed in an annular shape and that is configured to be mountable on a finger or a wrist, a sensor unit that is provided in the main body and is configured to measure biometric data including blood pressure, and a sensor-side short-range wireless communication unit that is configured to communicate with the portable control unit when the portable control unit is located within a predetermined range. The portable control unit includes an image capturing unit that is configured to capture an image, a display unit that is configured to prompt a user who grips the portable control unit with a hand on which the annular biometric sensor is mounted to capture an image of a face of the user, and to display an image captured by the image capturing unit, an inclination sensor that is configured to detect an inclination of the portable control unit from a vertical direction, a unit-side short-range wireless communication unit that is configured to communicate with the annular biometric sensor when the annular biometric sensor is located within a predetermined range, and a controller that is configured to determine whether or not the portable control unit is gripped by the hand on which the annular biometric sensor is mounted, in accordance with a wireless communication state with the annular biometric sensor, to determine whether or not a measurement posture of the user is appropriate from a position of the face of the user in the image, which is recognized from the image, and the inclination of the portable control unit from the vertical direction, to control the biometric data measurement system based on a determination result as to whether or not the portable control unit is gripped by the hand on which the annular biometric sensor is mounted, and a determination result as to whether or not the measurement posture of the user is appropriate, and to acquire the biometric data including the blood pressure.

According to the biometric data measurement system of the exemplary embodiment, since the annular biometric sensor in which the sensor unit formed in an annular shape is provided is mounted on the finger or the wrist, the contact pressure (pressure) with a measurement site is stable, and biometric data including blood pressure can be measured with high accuracy. Further, it is determined whether or not the portable control unit is gripped by the hand on which the annular biometric sensor is mounted, in accordance with the wireless communication state with the annular biometric sensor, and it is determined whether or not the measurement posture of the user is appropriate from the position of the face of the user in the image, which is recognized from the image, and the inclination of the portable control unit from the vertical direction. Therefore, it is possible to estimate the relative position between the portable control unit and the face from the position of the face of the user in the image and the inclination of the portable control unit, and to estimate the height of the portable control unit (annular biometric sensor mounted on the hand gripping the portable control unit) from the heart. The biometric data measurement system is controlled (e.g., biometric data including the blood pressure is measured) based on the determination result as to whether or not the portable control unit is gripped by the hand on which the annular biometric sensor is mounted, and the determination result as to whether or not the measurement posture of the user is appropriate. Thus, the biometric data including the blood pressure can be measured with higher accuracy. Furthermore, since a cuff is not used, excellent portability is obtained, and the biometric data including the blood pressure can be measured in a non-invasive manner.

According to the present invention, excellent portability is obtained, and biometric data including blood pressure can be measured, where the measurement value is influenced by a difference between the height of a measurement site and the height of a heart (that is, influenced by hydrostatic pressure), in a non-invasive manner with higher accuracy.

### BRIEF DESCRIPTION OF DRAWINGS

FIG. 1 illustrates an overall configuration of a biometric data measurement system according to an exemplary embodiment.

FIG. 2 is a block diagram illustrating a functional configuration of the biometric data measurement system according to the exemplary embodiment.

FIG. 3 illustrates an arrangement of a sensor-side short-range wireless communication unit and a photoplethysmographic sensor (light-emitting element and light-receiving element) in an annular biometric sensor.

FIG. 4 illustrates a mounting state of the annular biometric sensor and a gripping state of a portable control unit when blood pressure or the like is measured.

FIG. 5 illustrates an inclination of the portable control unit.

FIG. 6 illustrates an example of display of recommended ranges of a display position of a face and a display size of the face.

FIG. 7 illustrates how to obtain a difference in height between the annular biometric sensor and a heart of a user.

FIG. 8(a) illustrates an example of an image in which only a trunk is inclined to the right, and FIG. 8(b) illustrates an example of an image in which the trunk and the portable control unit are similarly inclined to the right.

FIG. 9 illustrates a method of measuring an inclination of the trunk of the user.

FIG. 10 is a flowchart illustrating a processing procedure of a process of measuring blood pressure and the like by the annular biometric sensor that includes the biometric data measurement system according to the exemplary embodiment.

FIG. 11 is a flowchart illustrating a processing procedure of a process of measuring blood pressure and the like by the portable control unit that includes constitutes the biometric data measurement system according to the exemplary embodiment.

### DETAILED DESCRIPTION OF EMBODIMENTS

Hereinafter, exemplary embodiments of the present invention will be described in detail with reference to the drawings. It is noted that, in the drawings, the same reference signs are used for the same or corresponding portions. Further, in each drawing, the same elements are denoted by the same reference signs, and a repetitive description will be omitted.

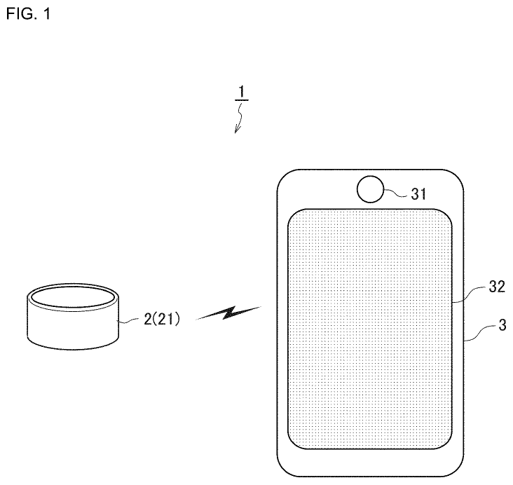

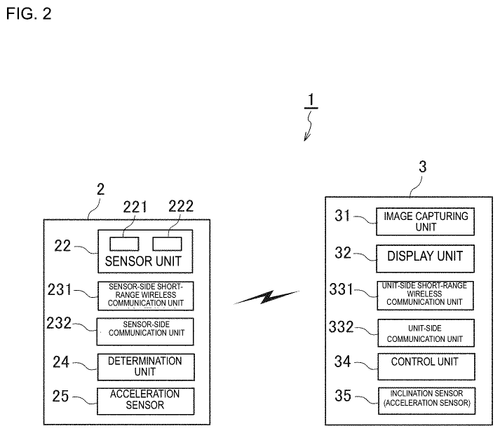

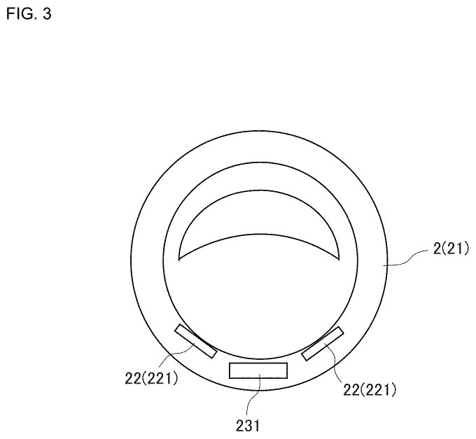

First, a configuration of a biometric data measurement system 1 according to an exemplary embodiment will be described with reference to FIGS. 1 to 3. FIG. 1 illustrates the overall configuration of a biometric data measurement system 1. FIG. 2 is a block diagram illustrating the functional configuration of the biometric data measurement system 1. FIG. 3 illustrates an arrangement of a sensor-side short-range wireless communication unit 231 and a photoplethysmographic sensor 22 (e.g., light-emitting element 221 and light-receiving element 222) in an annular biometric sensor 2.

The biometric data measurement system 1 mainly includes the annular biometric sensor 2 and a portable control unit 3, which are communicatively connected to each other via wireless communication. In particular, the biometric data measurement system 1 has excellent portability and is configured to measure biometric data including blood pressure, whose measurement value is influenced by a difference between the height of a measurement site and the height of a heart (that is, influenced by hydrostatic pressure), in a non-invasive manner with higher accuracy.

The annular biometric sensor 2 mainly includes a main body 21 formed in an annular shape (ring type or wristband type) such that it is configured to be mountable (i.e., configured or be mounted) on a finger or a wrist of a user, a sensor unit 22 that is provided on an inner side of the main body 21 and is configured to measure (e.g., detects) at least blood pressure, a sensor-side short-range wireless communication unit 231 that is configured to communicate with the portable control unit 3 when the portable control unit 3 is located within a predetermined range, a sensor-side communication unit 232 that has a communication range wider than the sensor-side short-range wireless communication unit 231, and transmits and receives data (e.g., measurement data, control data, and the like) to and from the portable control unit 3, a determination unit 24 that is configured to determine whether or not the annular biometric sensor 2 is mounted, and an acceleration sensor 25 that is configured to detect a body motion. According to an exemplary aspect, the annular biometric sensor 2 preferably includes a temperature sensor that detects a body surface temperature.

The portable control unit 3 mainly includes an image capturing unit 31 that is configured to capture an image (e.g., still image or moving image), a display unit 32 that performs presentation (e.g., display) to prompt the user who grips the portable control unit 3 by the hand on which the annular biometric sensor 2 is mounted to capture an image of a face of the user, and displays the image captured by the image capturing unit 31, a unit-side short-range wireless communication unit 331 that is configured to communicate with the annular biometric sensor 2 when the annular biometric sensor 2 is located within a predetermined range, a unit-side communication unit 332 that has a communication range wider than the unit-side short-range wireless communication unit 331, and transmits and receives data (e.g., measurement data, control data, and the like) to and from the annular biometric sensor 2, a controller 34 that determines (e.g., a “gripping determination”) whether or not the portable control unit 3 is gripped by the hand on which the annular biometric sensor 2 is mounted, in accordance with a short-range wireless communication state with the annular biometric sensor 2, determines (e.g., a “posture determination”) whether or not a measurement posture of the user is appropriate from a position of a face of the user in an image, which is recognized from the image, and an inclination of the portable control unit 3 from a vertical direction, controls the image capturing unit 31, the display unit 32, the unit-side short-range wireless communication unit 331, the unit-side communication unit 332, and the annular biometric sensor 2 based on a determination result (e.g., a “gripping determination result”) as to whether or not the portable control unit 3 is gripped by the hand on which the annular biometric sensor 2 is mounted, and a determination result (e.g., a “posture determination result”) as to whether or not the measurement posture of the user is appropriate, and acquires biometric data (e.g., biometric information) including blood pressure, and an inclination sensor (or acceleration sensor) 35 that is configured to detect an inclination of the portable control unit 3 from a vertical direction.

As the portable control unit 3, which is a control terminal, for example, a portable terminal such as a smartphone can be suitably used in an exemplary aspect. It is noted that, in the present embodiment, a smartphone is used as the portable control unit 3. Each component will be described below.

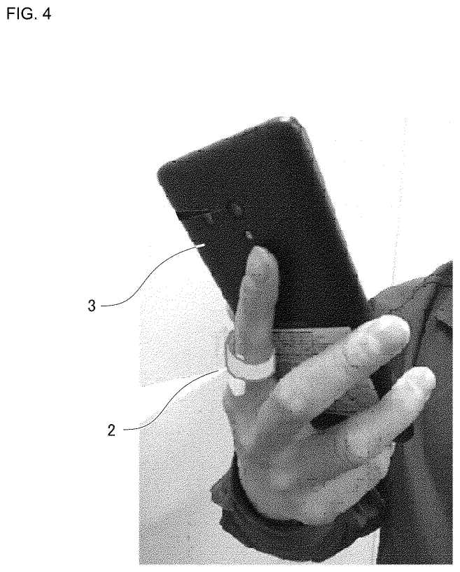

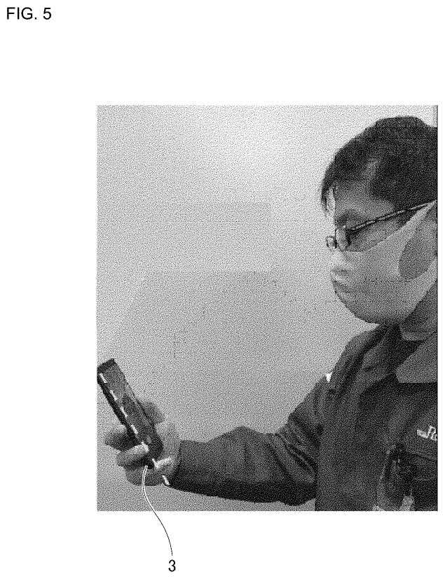

The main body 21 of the annular biometric sensor 2 is formed in an annular shape (ring type) so that the annular biometric sensor 2 can be mounted on a finger. Alternatively, the main body 21 is formed in an annular shape (wristband type) so that the annular biometric sensor 2 can be mounted on a wrist. Note that, in the present embodiment, a ring-type biometric sensor to be mounted on a finger will be described as an example of the annular biometric sensor 2. The annular biometric sensor 2 is mounted, for example, on the index finger of one hand (the right hand in the examples of FIGS. 4 to 7). The finger on which the annular biometric sensor 2 is mounted may be the middle finger, the ring finger, the little finger, or the thumb. It is also noted that the portable control unit 3 is gripped by the hand (e.g., the right hand in the examples of FIGS. 4 to 7) on which the annular biometric sensor 2 is mounted.

For example, the sensor unit 22 is a photoplethysmographic sensor that includes a light-emitting element (light-emitting unit) 221 and a light-receiving element (light-receiving unit) 222 and is configured to detect a photoplethysmographic signal. The photoplethysmographic sensor optically measures a pulse or the like by using the light absorption characteristics of hemoglobin in blood. In an exemplary aspect, the sensor unit 22 can also be referred to as the photoplethysmographic sensor 22 below. The sensor unit (photoplethysmographic sensor) 22 is provided on the inner side of the main body 21.

Further, according to an exemplary aspect, the sensor unit (photoplethysmographic sensor) 22 is preferably disposed in the main body 21 so as to be positioned (located) on a pad side of the finger of the user when the annular biometric sensor 2 is mounted on the finger. This is because pulse wave sensors, including the photoplethysmographic sensor 22, more easily acquire a biometric signal on the pad side of the finger than on the dorsal side of the finger. It is noted that, as illustrated in FIG. 3, when, if the sensor-side short-range wireless communication unit 231 is disposed so as to be positioned on the pad side of the finger, the position of the sensor-side short-range wireless communication unit 231 overlaps the sensor unit (photoplethysmographic sensor) 22, the sensor unit (photoplethysmographic sensor) 22 may be displaced so that the sensor unit (photoplethysmographic sensor) 22 is positioned on the side surface of the finger.

The sensor unit 22 is configured to measure (e.g., detects) at least blood pressure. In the present embodiment, a blood pressure sensor that estimates blood pressure from a photoplethysmographic waveform will be described as an example. As a method for estimating the blood pressure from the photoplethysmographic waveform, a known method (see, for example, Japanese Unexamined Patent Application Publication No. 2016-16295) can be used. That is, the annular biometric sensor 2 is a so-called cuffless sphygmomanometer that does not use a cuff. Note that, in addition, a blood pressure estimation technique (method) using a pulse wave transit time or the like may be used.

In general, a blood pressure measurement value obtained by any method may be inaccurate due to an influence of hydrostatic pressure. To avoid the influence of hydrostatic pressure, it is necessary that the blood pressure be measured at the height of the heart of the user or near the heart. When the blood pressure is measured above the height of the heart, the measurement result is too low. When the blood pressure is measured below the height of the heart, the measurement result is too high. When a difference in height between a blood pressure measurement position and the heart is 10 cm, an error of 7 to 8 mmHg is added to the blood pressure measurement value. In other words, when the blood pressure is measured with a finger in a state where the arm is loosely lowered, a height difference of about 50 cm occurs, and an error of 35 to 40 mmHg is added. When the blood pressure is measured by a general user who is not trained like medical personnel, the blood pressure is measured often at a height that is significantly different from the height of the heart of the user, and thus an error in the blood pressure measurement value is caused. Even a method for estimating the blood pressure from a photoplethysmographic waveform measured with a finger requires the influence of static pressure to be minimized or eliminated in order to accurately measure the blood pressure.

Also, a known method (see, for example, Japanese Patent Application No. 2017-506158) can be used as a method for estimating the blood glucose level from the photoplethysmographic waveform. Since the photoplethysmographic waveform is also influenced by the blood pressure value at this time, it also has an influence on the estimated blood glucose level. Therefore, an appropriate measurement posture is also needed for a blood glucose sensor in order to limit the influence of the blood pressure. In addition, a posture that compresses the abdomen, such as leaning forward, may increase the blood pressure, but the pulse and respiration may also change depending on the posture, and it may be necessary to take an appropriate measurement posture. Information on blood vessel resistance is also included in the photoplethysmographic waveform. When blood vessel resistance is obtained, the photoplethysmographic waveform is also influenced by the blood pressure, and thus variations can be reduced by performing measurement at the height of the heart. It is noted that although blood vessel resistance is used as an example, the same applies to a case of estimating a blood flow, a blood glucose level, and an artery solidification degree from waveforms. In addition, since the measurement posture has an influence on the pulse rate, the blood flow, the body surface temperature, and the respiration itself, measurement variations can be reduced by performing measurement in a predetermined posture. Here, the biometric data (e.g., biometric information) to be measured may include, for example, a pulse wave, a pulse, an oxygen saturation level, a blood glucose level, a body surface temperature, an amount of activity, blood vessel resistance, a blood flow, an artery solidification degree, and respiration, and the like, in addition to the blood pressure. As described above, by simultaneously measuring a plurality of pieces of biometric data (information), the physical condition, signs of disease, and the like can be accurately estimated.

The sensor-side short-range wireless communication unit 231 is configured to perform short-range wireless communication that allows communication with the portable control unit 3 when the portable control unit 3 is located within a predetermined range. For example, the sensor-side short-range wireless communication unit 231 includes a near field communication (NFC) module in an exemplary aspect. The portable control unit 3 in which the unit-side short-range wireless communication unit 331 (details will be described later) is built is gripped by a hand having the annular biometric sensor 2, in which the sensor-side short-range wireless communication unit (NFC module) 231 is built, mounted on a finger thereof. In this manner, the annular biometric sensor 2 (sensor-side short-range wireless communication unit 231) and the portable control unit 3 (unit-side short-range wireless communication unit 331) come close to each other, and short-range wireless communication (NFC communication) is possible between the annular biometric sensor 2 and the portable control unit 3.

As illustrated in FIG. 3, the sensor-side short-range wireless communication unit 231 is disposed in the main body 21 so as to be positioned (i.e., located) on the pad side of the finger of the user when the annular biometric sensor 2 is mounted on the finger. FIG. 3 illustrates the arrangement of the sensor-side short-range wireless communication unit (NFC module) 231 and the photoplethysmographic sensor 22 (light-emitting element 221 and light-receiving element 222) when viewed from an axial direction (and the tip side of the finger) of the annular biometric sensor 2. When the portable control unit 3 is gripped by the hand on which the annular biometric sensor 2 is mounted, the palm side comes into contact with the portable control unit 3. At this time, when the sensor-side short-range wireless communication unit (NFC module) 231 is positioned on the dorsal side of the finger, communication with the portable control unit 3 is performed with the finger interposed therebetween. Since the living body (finger) absorbs electromagnetic waves, such a layout is influenced by the finger. In contrast, when the sensor-side short-range wireless communication unit (NFC module) 231 is positioned on the pad side of the finger, it is less likely to be influenced by the finger, and the communication state will be more stable.

In an exemplary aspect, the sensor-side communication unit 232 uses a wireless communication method (wireless communication standard) having a communication range wider than NFC, and transmits and receives data (measurement data, control data, and the like) to and from the portable control unit 3. Here, in the present embodiment, Bluetooth® is used as the wireless communication standard. That is, the sensor-side communication unit 232 has a transmission function and a reception function based on Bluetooth®. Note that the wireless communication standard to be used is not limited to Bluetooth®, and other standards may be used. More specifically, the sensor-side communication unit 232 transmits mounting state information (details will be described later) of the annular biometric sensor 2 to the portable control unit 3. On the other hand, the sensor-side communication unit 232 receives a measurement (start) command transmitted from the portable control unit 3. The sensor-side communication unit 232 transmits the acquired biometric data such as the blood pressure to the portable control unit 3 (at a predetermined timing (or period)).

The determination unit 24 is configured to determine whether or not the annular biometric sensor 2 is mounted on the finger or the wrist. If posture determination (details will be described later) is performed when the annular biometric sensor 2 is not mounted, there is a concern that the posture is erroneously determined to be appropriate even though the posture is not appropriate. Such a problem can be avoided by performing posture determination only when the annular biometric sensor 2 is mounted.

Here, as a method of determining whether or not the annular biometric sensor 2 is mounted, it is desirable to determine whether or not the photoplethysmographic sensor 22 detects a pulse wave. This is because the probability of erroneously determining that the annular biometric sensor 2 is mounted even though the annular biometric sensor 2 is not mounted on the finger is low. However, since it is necessary to measure two or more beats to determine a pulse wave, it may take three seconds or more. Therefore, it may be determined whether or not the amount of light received by the photoplethysmographic sensor 22 exceeds a threshold value. When the photoplethysmographic sensor 22 is of a reflective type, the amount of received light is low if the annular biometric sensor 2 is not mounted. Thus, it is considered that the annular biometric sensor 2 is not mounted when the amount of received light lowers the threshold value. When the photoplethysmographic sensor 22 is of a transmissive type, the amount of received light is high if the annular biometric sensor 2 is not mounted. Thus, it is considered that the annular biometric sensor 2 is not mounted when the amount of received light exceeds the threshold value. This method makes it possible to perform determination in a short time. However, there is a concern that any object that blocks light is determined to be mounted (that is, an erroneous determination) even if the object is inserted into the annular biometric sensor 2. Therefore, in an exemplary aspect, a configuration can be used in which it is determined whether or not the annular biometric sensor 2 is mounted on the finger in combination with, for example, a method of determining that the annular biometric sensor 2 is not mounted when no movement is detected by the acceleration sensor 25, a gyro sensor, or the like, or a method of determining that the annular biometric sensor 2 is not mounted using a temperature sensor that detects the body surface temperature when the detected temperature is equal to or lower than a predetermined value.

Furthermore, a determination result by the determination unit 24 is transmitted from the sensor-side communication unit 232 to the portable control unit 3. The controller 34 of the portable control unit 3 can be configured to prohibit or prevent the posture determination of the user (details will be described later) when the annular biometric sensor 2 is not mounted on the finger or wrist of the hand.

The acceleration sensor 25 is configured to detect an acceleration of the annular biometric sensor 2, that is, the body motion of the user equipped with the annular biometric sensor 2. It is also noted that the detection result of the acceleration sensor 25 is transmitted from the sensor-side communication unit 232 to the portable control unit 3.

On the other hand, the image capturing unit (e.g., camera) 31 of the portable control unit 3 captures an image (e.g., still image or moving image). The image capturing unit 31 is provided on the surface of the portable control unit 3 on the display unit 32 side. As illustrated in FIGS. 4 to 7, the image capturing unit 31 captures an image of the face of the user gripping the portable control unit 3 with one hand (for example, the right hand) on which the annular biometric sensor 2 is mounted.

The display unit 32 is, for example, an LCD display. The display unit 32 displays (notifies) the following images, information, and the like (1) to (5), for example.

(1) The display unit 32 performs display (notification) to prompt the user to hold the portable control unit 3 with the hand on which the annular biometric sensor 2 is mounted.

(2) The display unit 32 performs display (e.g., a presentation) to prompt the user to capture an image of the face of the user with the portable control unit 3 such that the face of the user fits in the frame.

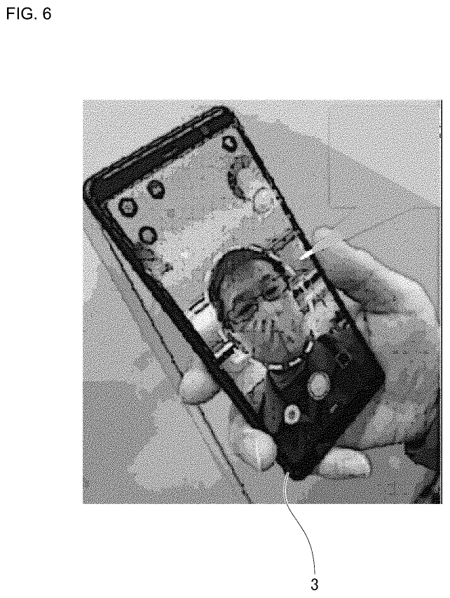

(3) The display unit 32 displays an image (e.g., still image or moving image) captured by the image capturing unit 31 in real time. In addition, the display unit 32 graphically displays (e.g., presents) the recommended ranges of the display position of the face and the display size of the face. More specifically, as illustrated in FIG. 6, the display unit 32 superimposes a figure such as a substantial ellipse or rectangle representing the appropriate position and size of the face. By graphically displaying the appropriate ranges of the display position of the face and the display size (size) of the face on the display unit 32 (display) as described above, the user can easily recognize the appropriate range. Therefore, the user can easily correct the position and size of the displayed face by himself/herself.

(4) When the controller 34 of the portable control unit 3 recognizes the face of the user in the image, the display unit 32 performs notification (e.g., display) of whether or not the display position of the face and the display size of the face are within the recommended ranges. As described above, since both the actual position and size (size) of the face in the image and the position and size (size) of the face within an appropriate range are displayed, correction by the user is facilitated. It is desirable to use the height of the eyes as the standard for the height of the face. When the position of the eyes is automatically determined by automatic face determination, the estimation accuracy of the relative position of the face and the heart can be improved.

(5) The display unit 32 performs display (e.g., notification) to prompt the user to make adjustments so that the trunk of the user is in the vertical direction, i.e., to provide an appropriate or sufficient measurement posture.

According to an exemplary aspect, the unit-side short-range wireless communication unit 331 is configured to perform short-range wireless communication that enables communication with the annular biometric sensor 2 when the annular biometric sensor 2 is within a predetermined range. For example, the unit-side short-range wireless communication unit 331 includes a near field communication (NFC) module. Therefore, the portable control unit 3 in which the unit-side short-range wireless communication unit (NFC module) 331 is built is gripped by a hand on which the annular biometric sensor 2 in which the sensor-side short-range wireless communication unit (NFC module) 231 is built is mounted on a finger. In this manner, the annular biometric sensor 2 (sensor-side short-range wireless communication unit 231) and the portable control unit 3 (unit-side short-range wireless communication unit 331) come close to each other, and short-range wireless communication (NFC communication) is possible between the annular biometric sensor 2 and the portable control unit 3.

Therefore, the controller 34 of the portable control unit 3 can determine whether or not the portable control unit 3 is gripped by the hand on which the annular biometric sensor 2 is mounted, in accordance with whether or not NFC communication (short-range wireless communication) with the annular biometric sensor 2 is possible. That is, when NFC communication (short-range wireless communication) with the annular biometric sensor 2 is possible, the controller 34 of the portable control unit 3 determines that the portable control unit 3 is gripped by the hand on which the annular biometric sensor 2 is mounted. On the other hand, when the NFC communication with the annular biometric sensor 2 is disabled, the controller 34 of the portable control unit 3 determines that the portable control unit 3 is not gripped by the hand on which the annular biometric sensor 2 is mounted. As described above, by using NFC, it can easily be determined whether or not the annular biometric sensor 2 is mounted on the hand gripping the portable control unit 3. In addition, an erroneous blood pressure value or the like can be prevented from being measured in a state where the annular biometric sensor 2 is not mounted.

It is noted that the sensor-side short-range wireless communication unit 231 and the unit-side short-range wireless communication unit 331 can each be composed of a Bluetooth® module instead of the NFC module in an alternative aspect. In this case, the controller 34 of the portable control unit 3 determines whether or not the portable control unit 3 is gripped by the hand on which the annular biometric sensor 2 is mounted, in accordance with whether or not the received signal strength indicator of radio waves transmitted from the annular biometric sensor 2 is equal to or greater than a predetermined value.

Since Bluetooth® generally allows communication even at a distance of 10 meters or more, it is not possible to determine whether or not the portable control unit 3 is gripped by the hand on which the annular biometric sensor 2 is mounted, only by whether or not communication is possible. Therefore, when the received signal strength indicator (RSSI) in the portable control unit 3 is equal to or greater than or the predetermined value, it is determined that the portable control unit 3 is gripped by the hand on which the annular biometric sensor 2 is mounted. The RSSI value fluctuates greatly depending on the environment, obstacles, and the like. Therefore, when the sensor-side short-range wireless communication unit 231 is disposed on the dorsal side of the finger, communication is performed through the finger, and there is a concern that the RSSI is not stable due to the influence of the size of the finger (individual difference) and mounting condition. Thus, in order to stabilize the communication state, when the annular biometric sensor 2 is mounted on the finger, it is desirable that the sensor-side short-range wireless communication unit 231 (e.g., Bluetooth® module) be disposed in the main body 21 so as to be positioned on the pad side of the finger.

In this case, by using Bluetooth® modules for the sensor-side short-range wireless communication unit 231 and the unit-side short-range wireless communication unit 331, it is possible to perform (both) determination of whether or not the portable control unit 3 is gripped by the hand on which annular biometric sensor 2 is mounted and data communication (that is, to perform both together) only using Bluetooth® modules.

The unit-side communication unit 332 uses a wireless communication method (wireless communication standard) having a communication range wider than NFC, and transmits and receives data (control data (command), measurement data, and the like) to and from the annular biometric sensor 2 (sensor-side communication unit 232). As described above, in the present embodiment, Bluetooth® is used as the wireless communication standard. That is, the unit-side communication unit 332 has a transmission function and a reception function based on Bluetooth® according to an exemplary aspect. More specifically, the unit-side communication unit 332 can be configured to transmit a measurement (start) command to the sensor-side communication unit 232. On the other hand, the unit-side communication unit 332 can receive mounting state information transmitted from the annular biometric sensor 2. Also, the unit-side communication unit 332 receives biometric data such as blood pressure transmitted from the annular biometric sensor 2.

The controller 34 determines (e.g., a “gripping determination”) whether or not the portable control unit 3 is gripped by the hand on which the annular biometric sensor 2 is mounted, in accordance with the wireless communication state with the annular biometric sensor 2, determines (e.g., a “posture determination”) whether or not the measurement posture of the user is appropriate (e.g., is sufficient) from the position of the face of the user in the image, which is recognized from the image, and the inclination of the portable control unit 3 from the vertical direction, controls the image capturing unit 31, the display unit 32, the unit-side short-range wireless communication unit (NFC module) 331, the unit-side communication unit 332, and the annular biometric sensor 2 based on the determination result (e.g., a “gripping determination result”) as to whether or not the portable control unit 3 is gripped by the hand on which the annular biometric sensor 2 is mounted, and the determination result (e.g., a “posture determination result”) as to whether or not the measurement posture of the user is appropriate, and acquires biometric data (e.g., biometric information) including the blood pressure. Therefore, in an exemplary aspect, the controller 34 can include a microprocessor that performs calculations, an EEPROM that stores programs and the like for causing the microprocessor to execute various processes, a RAM that temporarily stores data, an external interface (I/F), and the like. According to an exemplary aspect, each function and operation of the controller 34 is implemented by the microprocessor executing a program stored in the EEPROM or the like.

The controller 34 statistically estimates the size of the face and the distance between the face and the heart from the height. Then, the controller 34 obtains the distance between the face and the portable control unit 3 from the size of the face. Subsequently, when the trunk of the user is not inclined, the controller 34 regards the distance between the face and the heart as the difference in height between the face and the heart. The controller 34 obtains the difference in height between the portable control unit 3 (annular biometric sensor 2) and the heart based on the difference in height between the face and the heart and the distance between the face and the portable control unit 3.

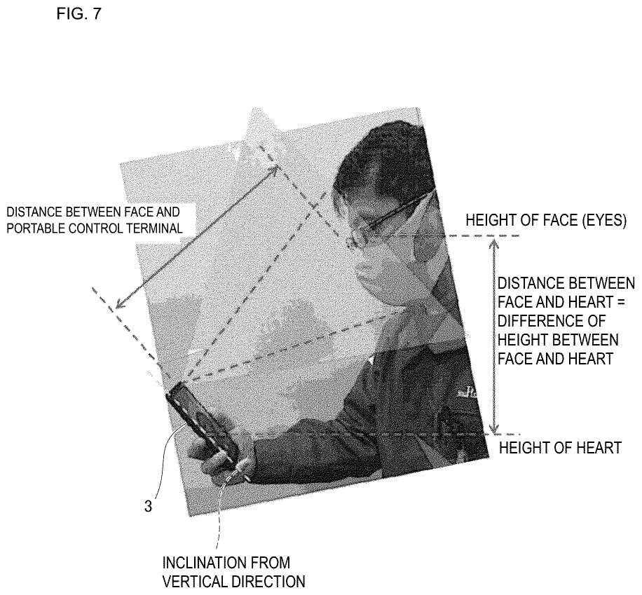

More specifically, the controller 34 statistically estimates the relative position of the face and the heart from the height. However, since the relative position shifts in a posture in which the trunk is greatly bent, such as leaning forward, it is assumed here that the trunk is not inclined at a sitting position. For example, “AIST human body size database 1991-1992” does not have data on the height of the heart; therefore, in the present embodiment, data on the nipple height is used instead. The difference in height between the face and the heart can be obtained statistically by using B2 inner canthus height-B6 nipple height instead. The controller 34 estimates, for example, the difference (see FIG. 7) between the eyes (inner canthus height) and the nipple (nipple height) from statistical data, from the size (total head height) of the face in the image.

As described above, when the face is displayed at a predetermined position on the display unit (display) 32, a direction in which the face is located is determined from the image capturing unit (camera) 31 of the portable control unit 3. When the face is displayed with a predetermined size on the display unit 32, it is possible to estimate the distance (see FIG. 7) between the portable control unit 3 and the face. Since the size of the face varies from person to person, the estimation accuracy of the distance between the portable control unit 3 and the face is improved by statistically estimating the size of the face from the physical information of the user (for example, the height and the weight). The physical information (height and the like) of the user, which is input to the portable control unit 3 in advance by the user and then is stored in a memory or a server may be read, or data such as health checkup stored in the server may be read.

As described above, when the size of the face can be estimated, and the face is displayed at a predetermined position and has a predetermined size on the display unit 32, the relative position of the portable control unit 3 to the face is determined. The inclination of the portable control unit 3 is obtained from an inclination sensor (e.g., an acceleration sensor) 35. The absolute position of the portable control unit 3 with respect to the face is determined from the relative position of the portable control unit 3 to the face and the inclination of the portable control unit 3 (because the vertical direction is determined). As for the relative position of the portable control unit 3 and the annular biometric sensor 2, the annular biometric sensor 2 is located approximately at the center of the back side of the portable control unit 3 according to the gripping method as illustrated in FIG. 4. Since the vertical direction can be determined from the inclination of the portable control unit 3, the absolute positions of the portable control unit 3 and the annular biometric sensor 2 can be accurately estimated.

When the trunk of the user is not inclined, the distance between the face and the heart can be regarded as the difference in height between the face and the heart. The controller 34 obtains the difference in height between the annular biometric sensor 2 and the heart based on the difference in height between the face and the heart, the absolute positions of the face and the portable control unit 3, and the absolute positions of the portable control unit 3 and the annular biometric sensor 2. That is, the controller 34 obtains (see FIG. 7) the difference in height between the portable control unit 3 and the heart based on the difference in height between the face and the heart, the distance between the face and the portable control unit 3, and the inclination of the portable control unit 3, and further obtains the difference in height between the annular biometric sensor 2 and the heart based on the absolute positions of the portable control unit 3 and the annular biometric sensor 2.

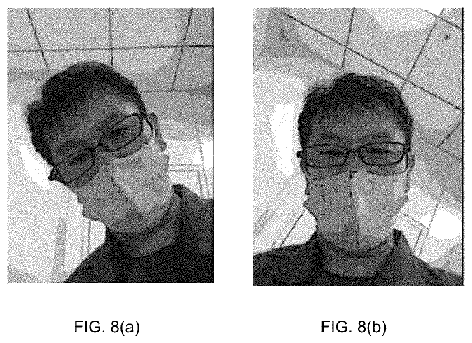

It is noted that, of the inclination of the trunk of the user, an inclination in a right-left direction can be estimated from the inclination of the face in the image in the right-left direction and the inclination of the portable control unit 3 in the right-left direction. When the right-left inclination exceeds a predetermined range, the user is notified through the display unit 32 or the like. Here, FIG. 8(a) illustrates an example of an image in which only the trunk is inclined to the right. FIG. 8(b) illustrates an example of an image in which the trunk and the portable control unit 3 are similarly inclined to the right.

Furthermore, the portable control unit 3 includes an inclination sensor (or acceleration sensor) 35 that is configured to detect the inclination of the device (portable control unit 3) from the vertical direction. The controller 34 determines whether or not the inclination of the trunk of the user with respect to the vertical direction and the right-left direction is within a predetermined range, based on the inclination of the portable control unit 3 from the vertical direction, which is detected by the inclination sensor 35.

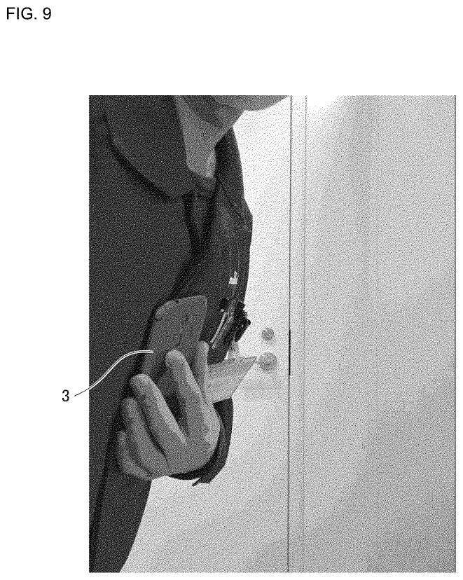

Further, as illustrated in FIG. 9, it is possible to recognize the inclination of the trunk of the user in a front-rear direction in a manner that the inclination sensor (acceleration sensor) 35 detects the inclination of the portable control unit 3 from the vertical direction when the portable control unit 3 is in close contact with the trunk of the user.

At this time, the controller 34 of the portable control unit 3 determines whether or not the inclination of the trunk of the user (with respect to the vertical direction and the front-rear direction) is within a predetermined range, based on the acquired (detected) inclination of the portable control unit 3 from the vertical direction. The display unit 32 of the portable control unit 3 displays the determination result by the controller 34. In this case, by bringing the portable control unit 3 into close contact with the trunk and measuring the inclination of the portable control unit 3 at this time, the inclination of the trunk from the vertical direction can be determined. Thus, the user can be notified of leaning forward or warping back and such that the user can perform a corrective action.

It is also noted that, when the difference in height between the annular biometric sensor 2 and the heart of the user is out of the predetermined range, and when the inclination of the trunk of the user from the vertical direction is out of the predetermined range, the controller 34 of the portable control unit 3 determines that the measurement posture of the user is not appropriate or sufficient. The inclination of the trunk from the vertical direction causes deviations in the estimated values of the heights of the face and the heart. By determining whether or not the inclination is out of the predetermined range, it can be understood whether or not the measured blood pressure value deviates from the true value.

When the user leans forward or warps back (e.g., leans back) during measurement, the positional relationship between the heights of the face and the heart is shifted, resulting in an underestimation of the height of the heart compared to the actual height of the heart, and thus the accuracy of the blood pressure value decreases. In addition, postures that compress the abdomen, such as leaning forward, may increase the blood pressure. However, it is difficult for the user to perceive such leaning forward or warping back. By determining the inclination of the trunk from the vertical direction, the user can be notified of leaning forward or warping back and can therefore perform a corrective action.

As described above, it is important to measure the blood pressure at the height of the heart at rest. Thus, when measurement is not performed in an appropriate or sufficient posture, it is not possible to measure the accurate blood pressure value. On the other hand, the measurement posture of the user is limited (restricted) by performing measurement at the height of the heart. Thus, measurement may be difficult when continuous or periodic data is required. Therefore, it is important to calculate the reliability of the measurement value and to perform correction to be approximately equal to the blood pressure value in the case of an appropriate measurement posture. Since the blood pressure value becomes inaccurate as the posture deviates from the appropriate posture, by calculating the reliability of the measurement value in accordance with the deviation from the appropriate posture, the user can handle the measurement value in consideration of the risk that the blood pressure measurement value deviates from the true value.

Therefore, in the exemplary aspect, the controller 34 is configured to calculate or determine the reliability of biometric data including the (measured) blood pressure based on the determination result of the inclination of the trunk of the user (posture determination result). Since it is important to measure blood pressure at the height of the heart at rest, it is not possible to measure the accurate blood pressure value when the measurement is not performed in the appropriate posture. The blood pressure value becomes inaccurate as the posture deviates from an appropriate posture, but by calculating the reliability, it is possible to handle the measurement value in consideration of the risk that the blood pressure measurement value deviates from the true value.

In addition, if the measured blood pressure value is corrected to be approximately equal to the blood pressure value in the case of an appropriate measurement posture, it becomes more convenient for the user. When the absolute positions of the face and the portable control unit 3 can be estimated, the deviation in height between the annular biometric sensor 2 and the heart can be estimated. Thus, the blood pressure value correction corresponding to the deviation in height may be performed. Further, the controller 34 may correct biometric data such as blood pressure based on the determination result of the inclination of the trunk of the user (posture determination result). For example, leaning forward may increase the blood pressure. Thus, in the case of leaning forward, the estimated value may be corrected to be lower from the inclination of the trunk and the blood pressure value data obtained in advance.

When the difference in height between the annular biometric sensor 2 and the heart can be estimated, the blood pressure value can be corrected. However, measurement in which the annular biometric sensor 2 is located at the (vertical) height of the heart improves the blood pressure estimation accuracy. That is, the blood pressure accuracy is more stable when the blood pressure is measured at the height of the heart each time than when the blood pressure is measured at a position lower or higher than the heart. However, measurement at the height of the heart limits the measurement posture of the user; therefore, it may be difficult when continuous or periodic data is required (there is a concern that pain is given to the user). Therefore, by correcting the measured blood pressure value to be approximately equal to the blood pressure value in the case of an appropriate measurement posture, continuous data and periodic data can be accurately acquired.

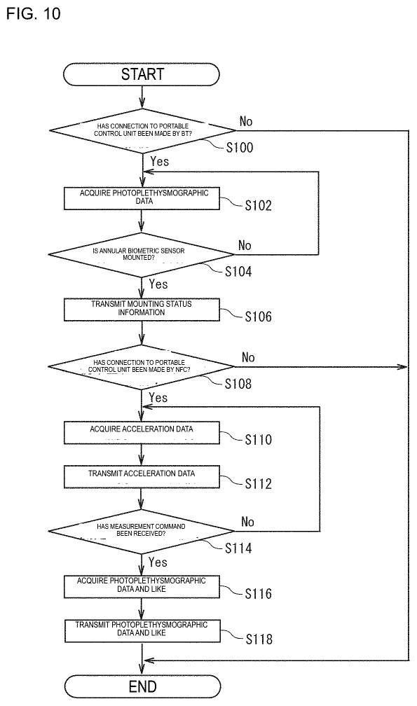

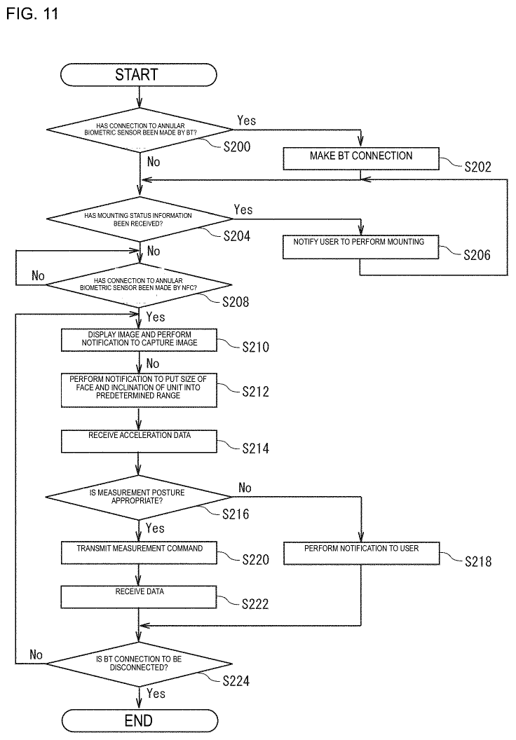

Next, the operation of the biometric data measurement system 1 will be described with reference to FIGS. 10 and 11. FIG. 10 is a flowchart illustrating a processing procedure of a process or method of measuring blood pressure and the like by the annular biometric sensor 2 that includes the biometric data measurement system 1. FIG. 11 is a flowchart illustrating a processing procedure of a process or method of measuring blood pressure and the like by the portable control unit 3 that includes the biometric data measurement system 1. The process illustrated in FIG. 10 is repeatedly executed mainly by the annular biometric sensor 2 at predetermined timings. The process illustrated in FIG. 11 is repeatedly executed mainly by the portable control unit 3 at predetermined timings.

First, the operation of the annular biometric sensor 2 (e.g., a process of measuring blood pressure and the like) will be described with reference to FIG. 10. In step S100, it is determined whether or not a connection to the portable control unit 3 has been made by Bluetooth®. Here, when a connection to the portable control unit 3 has not been made, this processing is temporarily exited. On the other hand, when a connection to the portable control unit 3 has been made, the process proceeds to step S102.

In step S102, a photoplethysmographic signal is acquired. Then, in step S104, it is determined whether or not the annular biometric sensor 2 is mounted on the finger based on the photoplethysmographic signal acquired in step S102. Here, when the annular biometric sensor 2 is not mounted on the finger, the process proceeds to step S102, and the processes of step S102 to S104 described above are repeatedly executed until the annular biometric sensor 2 is mounted on the finger. On the other hand, when the annular biometric sensor 2 is mounted on the finger, the process proceeds to step S106.

In step S106, information (e.g., mounting state information) indicating that the annular biometric sensor 2 is mounted on the finger is transmitted to the portable control unit 3.

In step S108, it is determined whether or not communication with the portable control unit 3 is possible by near field communication (NFC). Here, when communication with the portable control unit 3 is not possible by near field communication (NFC), this processing is temporarily exited. On the other hand, when communication with the portable control unit 3 is possible by near field communication (NFC), the process proceeds to step S110.

In step S110, acceleration data (body motion data) is acquired. The acquired acceleration data (body motion data) is transmitted to the portable control unit 3 in step S112.

Subsequently, in step S114, it is determined whether or not a measurement (start) command has been received from the portable control unit 3. Here, when a measurement (start) command has not been received, the process proceeds to step S110, and the processes of step S110 to S114 described above are repeatedly executed until the measurement (start) command is received. On the other hand, when a measurement (start) command has been received, the process proceeds to step S116.

In step S116, photoplethysmographic data (e.g., blood pressure data) and acceleration data (e.g., body motion data) are acquired. Then, in step S118, the photoplethysmographic data (e.g., blood pressure data) and acceleration data (e.g., body motion data) acquired in step S116 are transmitted to the portable control unit 3. Then, this processing is temporarily exited.

Next, the operation of the portable control unit 3 (process of measuring blood pressure and the like) will be described with reference to FIG. 11. In step S200, it is determined whether or not a connection to the annular biometric sensor 2 has been made by Bluetooth®. Here, when a connection to the annular biometric sensor 2 has not been made, in step S202, a connection (pairing) to the annular biometric sensor 2 is established by Bluetooth®, and then the process proceeds to step S204. On the other hand, when a connection to the annular biometric sensor 2 has been made, the process proceeds to step S204.

In step S204, it is determined whether or not information (e.g., mounting state information) indicating that the annular biometric sensor 2 is mounted on the finger has been received from the annular biometric sensor 2. Here, when the mounting state information has not been received, in step S206, information for prompting the user to mount the annular biometric sensor 2 is displayed (notified), and then the process proceeds to step S204. It is determined again whether or not the mounting state information has been received. On the other hand, when the mounting state information has been received, the process proceeds to step S208.

In step S208, it is determined whether or not communication with the annular biometric sensor 2 is possible by near field communication (NFC). Here, when communication with the annular biometric sensor 2 by near field communication (NFC) is not possible, this processing is repeatedly executed until communication with the annular biometric sensor 2 by near field communication (NFC) becomes possible. On the other hand, when communication with the annular biometric sensor 2 is possible by near field communication (NFC), the process proceeds to step S210.

In step S210, an image captured by the image capturing unit (camera) 31 is displayed, and information for prompting the user to capture an image of the user is displayed (notified).

Then, in step S212, information for prompting the user to put the size of the face in the image and the inclination of the portable control unit 3 within a predetermined range (appropriate range) is displayed (notified).

Subsequently, in step S220, the acceleration data (e.g., body motion data) transmitted from the annular biometric sensor 2 is received (acquired). In step S216, it is determined whether or not the measurement posture is within an appropriate range and whether or not the body motion is within an appropriate range. Here, when the measurement posture and the body motion are not within the appropriate ranges, in step S218, information for prompting the user to put the measurement posture and body motion within appropriate ranges is displayed (notification). Then, the process proceeds to step S224. On the other hand, when the measurement posture and body motion are within appropriate ranges, the process proceeds to step S220. It is also noted that the method of recognizing or determining whether or not the measurement posture is within the appropriate range is as described above, and thus a detailed description will be omitted here.

In step S220, a measurement (start) command for an instruction to start measurement is transmitted to the annular biometric sensor 2. Then, in step S222, the photoplethysmographic data (blood pressure data) and the acceleration data (body motion data) transmitted from the annular biometric sensor 2 are received (acquired). Here, the blood pressure, the blood glucose level, the pulse, the oxygen saturation level, and respiration are acquired from the photoplethysmographic data. The amount of activity and the inclination of the annular biometric sensor 2 are acquired from the acceleration data. In addition, when a temperature sensor is provided, the body surface temperature is acquired from the temperature data. Then, the process proceeds to step S224.

In step S224, it is determined whether or not the connection to the annular biometric sensor 2 by Bluetooth® is to be disconnected. Here, when the connection is to be disconnected, the connection to the annular biometric sensor 2 by Bluetooth® is disconnected, and then this processing is temporarily exited. On the other hand, when the connection is not to be disconnected, the process proceeds to step S210, and the processes of steps S210 to S224 described above are repeatedly executed.

As described above in detail, according to the present embodiment, since the annular biometric sensor 2 in which the sensor unit 22 formed in an annular shape is provided is mounted on the finger or the wrist, the contact pressure (pressure) with the measurement site is stable, and biometric data including blood pressure can be measured with high accuracy. Further, it is determined (gripping determination) whether or not the portable control unit 3 is gripped by the hand on which the annular biometric sensor 2 is mounted, in accordance with the short-range wireless communication state with the annular biometric sensor 2 (whether or not communication is possible), and it is determined (e.g., posture determination) whether or not the measurement posture of the user is appropriate from the position of the face of the user in the image, which is recognized from the image, and the inclination of the portable control unit 3 from the vertical direction. Therefore, it is possible to estimate the relative position between the portable control unit 3 and the face from the position of the face of the user in the image and the inclination of the portable control unit 3, and to estimate the height of the portable control unit 3 (annular biometric sensor 2 mounted on the hand gripping the portable control unit 3) from the heart.

According to the exemplary aspect, the annular biometric sensor 2 is controlled (biometric data including the blood pressure is measured) based on the determination result (e.g., a “gripping determination result”) as to whether or not the portable control unit 3 is gripped by the hand on which the annular biometric sensor 2 is mounted, and the determination result (e.g., a “posture determination result”) as to whether or not the measurement posture of the user is appropriate. Thus, the biometric data including the blood pressure can be measured with higher accuracy.

Furthermore, since a cuff is not used, excellent portability is obtained, and the biometric data including the blood pressure can be measured in a non-invasive manner. As a result, according to the present embodiment, excellent portability is obtained, and biometric data including blood pressure can be measured, whose measurement value is influenced by a difference between the height of a measurement site and the height of a heart (that is, influenced by hydrostatic pressure), in a non-invasive manner with higher accuracy.

At this time, according to the present embodiment, the face of the user in the image is automatically recognized, and the position of the heart of the user in the image is estimated based on the display position and the display size of the face. As described above, since the distance between the face and the heart can be estimated from the size of the face, the accuracy is improved for determining whether or not the annular biometric sensor 2 is located at the height of the heart.

It is noted that although an exemplary embodiment of the present invention has been described above, it is noted that the present invention is not limited to the above embodiment and various modifications are possible. For example, in the above-described embodiment, the configuration in which data (e.g., measurement data) such as measured blood pressure is sequentially transmitted to the portable control unit 3 is made, but a configuration in which the measurement data is stored in the EEPROM or RAM of the annular biometric sensor 2 and is read later (after measurement) may be used.

In the above exemplary embodiment, a photoplethysmographic sensor is used as the annular biometric sensor 2 (sensor unit 22), but the annular biometric sensor 2 (sensor unit 22) is not limited to a photoplethysmographic sensor in alternative aspects.

In the above exemplary embodiment, NFC is used for short-range wireless communication for determining whether or not the portable control unit 3 is gripped by the hand on which the annular biometric sensor 2 is mounted. A short-range wireless communication method other than NFC may be used. Further, it is noted that Bluetooth® is used as a wireless communication standard for transmitting and receiving data (control data (commands), measurement data, and the like) between the annular biometric sensor 2 and the portable control unit 3 according to an exemplary aspect. However, in other aspects, for example, BLE (Bluetooth® Low Energy) or the like may be used.

### REFERENCE SIGNS LIST

1 biometric data measurement system
2 annular biometric sensor
21 main body
22 sensor unit (photoplethysmographic sensor)
221 light-emitting element (light-emitting unit)
222 light-receiving element (light-receiving unit)
231 sensor-side short-range wireless communication unit (NFC module)
232 sensor-side communication unit (BT module)
24 determination unit
25 acceleration sensor
3 portable control unit
31 image capturing unit
32 display unit
331 unit-side short-range wireless communication unit (NFC module)
332 unit-side communication unit (BT module)
34 controller
35 inclination sensor (acceleration sensor)

1. A biometric data measurement system comprising:
an annular biometric sensor that includes:
a main body having an annular shape so as to be mountable on a finger or a wrist of a user,
a sensor unit in the main body and configured to measure biometric data including blood pressure, and
a sensor-side short-range wireless communication unit; and
a portable control unit configured to communicate with the biometric sensor and including:
an image capturing unit configured to capture an image,
a display unit configured to prompt the user gripping the portable control unit with a hand on which the annular biometric sensor is mounted to capture an image of a face of the user, and to display an image captured by the image capturing unit,
an inclination sensor configured to detect an inclination of the portable control unit from a vertical direction,
a unit-side short-range wireless communication unit configured to communicate with the annular biometric sensor when the annular biometric sensor is located within a predetermined range, and
a controller configured to:
determine whether or not the portable control unit is gripped by the hand on which the annular biometric sensor is mounted,
determine, based on a wireless communication state with the annular biometric sensor, whether or not a measurement posture of the user is sufficient from a position of the face of the user in the image, and the inclination of the portable control unit from the vertical direction,
control the biometric data measurement system based on a determination result as to whether or not the portable control unit is gripped by the hand on which the annular biometric sensor is mounted, and a determination result as to whether or not the measurement posture of the user is sufficient, and
acquire the biometric data including the blood pressure.

2. The biometric data measurement system according to claim 1, wherein the sensor-side short-range wireless communication unit is configured to communicate with the portable control unit when the portable control unit is located within a predetermined range.

3. The biometric data measurement system according to claim 1, wherein:
each of the sensor-side short-range wireless communication unit and the unit-side short-range wireless communication unit includes a near field communication (NFC) module, and
the controller of the portable control unit is configured to determine whether or not the portable control unit is gripped by the hand on which the annular biometric sensor is mounted based on whether or not NFC communication with the annular biometric sensor is possible.

4. The biometric data measurement system according to claim 1, wherein:
each of the sensor-side short-range wireless communication unit and the unit-side short-range wireless communication unit includes a Bluetooth module, and
the controller of the portable control unit is configured to determine whether or not the portable control unit is gripped by the hand on which the annular biometric sensor is mounted based on whether or not a received signal strength indicator of radio waves transmitted from the annular biometric sensor is equal to or greater than a predetermined value.

5. The biometric data measurement system according to claim 1, wherein the sensor-side short-range wireless communication unit is disposed in the main body and is configured to be positioned on a pad side of a finger of the user when the annular biometric sensor is mounted on the finger.

6. The biometric data measurement system according to claim 1, wherein the controller of the portable control unit is configured to acquire prestored physical information of the user, and to determine whether or not the measurement posture of the user is sufficient based on the acquired prestored physical information.

7. The biometric data measurement system according to claim 1, wherein the controller of the portable control unit is configured to determine that the measurement posture of the user is not sufficient when a difference in a height between the annular biometric sensor and a heart of the user is out of a predetermined range, and when an inclination of a trunk of the user from the vertical direction is out of a predetermined range.

8. The biometric data measurement system according to claim 1,
wherein the display unit is configured to graphically display recommended ranges of a display position of the face and a display size of the face. and
wherein:
the controller is configured to recognize the face of the user in the image, and
the display unit is configured to perform a notification as to whether or not the display position of the face and the display size of the face are within the recommended ranges.

9. The biometric data measurement system according to claim 7, wherein the display unit is configured to present whether or not each of the inclination of the portable control unit and the inclination of the trunk of the user is within a predetermined range.

10. The biometric data measurement system according to claim 7, wherein the annular biometric sensor further includes:
a determination unit configured to determine whether or not the annular biometric sensor is mounted on the finger or the wrist of the user, and
a sensor-side communication unit configured to transmit and receive data to and from the portable control unit.

11. The biometric data measurement system according to claim 10, wherein:
the sensor-side communication unit is configured to transmit a determination result as to whether or not the annular biometric sensor is mounted on the finger or the wrist of the user, and
the controller is configured to prevent a determination of the measurement posture of the user when the annular biometric sensor is not mounted on the finger or the wrist of the user according to the determination result.

12. The biometric data measurement system according to claim 1, wherein the biometric data further includes at least one of a blood glucose level, a pulse, respiration, a pulse wave, an oxygen saturation level, a body surface temperature, and an amount of activity of the user.

13. The biometric data measurement system according to claim 7, wherein the controller is further configured to calculate a reliability of the acquired biometric data based on the determination result of the measurement posture of the user.

14. The biometric data measurement system according to claim 7, wherein the controller is configured to correct the biometric data based on the determination result of the measurement posture of the user.

15. The biometric data measurement system according to claim 7, wherein the inclination sensor is configured to detect the inclination of the portable control unit from the vertical direction when the portable control unit is in close contact with the trunk of the user.

16. The biometric data measurement system according to claim 15, wherein the controller is configured to determine whether or not the inclination of the trunk of the user is within a predetermined range based on the detected inclination of the portable control unit from the vertical direction.

17. The biometric data measurement system according to claim 16, wherein the display unit is configured to display a determination result by the controller.

18. A biometric data measurement system comprising:
an annular biometric sensor that includes:
a main body configured to be mounted on a finger or a wrist of a user,
a sensor unit configured to measure a blood pressure of the user, and
a sensor-side short-range wireless communication unit; and
a portable control unit configured to communicate with the biometric sensor and including:
an image capturing unit configured to capture an image,
a display unit configured to prompt the user gripping the portable control unit with a hand on which the annular biometric sensor is mounted to capture and display an image of the user's face captured by the image capturing unit,
an inclination sensor configured to detect an inclination of the portable control unit from a vertical direction,
a unit-side short-range wireless communication unit configured to communicate with the annular biometric sensor when the annular biometric sensor is located within a predetermined range, and
a controller configured to:
determine whether or not the portable control unit is gripped by the hand on which the annular biometric sensor is mounted,
determine, based on a wireless communication state with the annular biometric sensor, whether or not a measurement posture of the user is sufficient from a position of the face of the user in the image, and the inclination of the portable control unit from the vertical direction,
control the biometric data measurement system based on: (i) a determination result as to whether or not the portable control unit is gripped by the hand on which the annular biometric sensor is mounted, and (ii) a determination result as to whether or not the measurement posture of the user is sufficient, and
acquire the biometric data including the blood pressure.

19. The biometric data measurement system according to claim 18, wherein the sensor-side short-range wireless communication unit is configured to communicate with the portable control unit when the portable control unit is located within a predetermined range.

20. The biometric data measurement system according to claim 18, wherein:
each of the sensor-side short-range wireless communication unit and the unit-side short-range wireless communication unit includes a near field communication (NFC) module, and
the controller of the portable control unit is configured to determine whether or not the portable control unit is gripped by the hand on which the annular biometric sensor is mounted based on whether or not NFC communication with the annular biometric sensor is possible.
